(19) United States
(12) Patent Application Publication
(10) Pub. No.: US 2023/0384312 A1
(43) Pub. Date: Nov. 30, 2023

(54) CIRCULATING MICROVESICLES EXPRESSING CARBONIC ANHYDRASE 9 FOR THE PROGNOSIS OF RENAL CELL CARCINOMA

(71) Applicants: CENTRE NATIONAL DE LA RECHERCHE SCIENTIFIQUE, Paris (FR); CENTRE HOSPITALIER UNIVERSITAIRE D'ANGERS, Angers (FR); UNIVERSITE D'ANGERS, Angers (FR); INSERM (INSTITUT NATIONAL DE LA SANTÉ ET DE LA RECHERCHE MÉDICALE), Paris (FR)

(72) Inventors: Maria del Carmen MARTINEZ, Tierce (FR); Pierre BIGOT, Avrillé (FR); Luisa VERGORI, Avrillé (FR)

(73) Assignees: CENTRE NATIONAL DE LA RECHERCHE SCIENTIFIQUE, Paris (FR); CENTRE HOSPITALIER UNIVERSITAIRE D'ANGERS, Angers (FR); UNIVERSITE D'ANGERS, Angers (FR); INSERM (INSTITUT NATIONAL DE LA SANTÉ ET DE LA RECHERCHE MÉDICALE), Paris (FR)

(21) Appl. No.: 18/249,830

(22) PCT Filed: Oct. 22, 2021
(86) PCT No.: PCT/EP2021/079371

§ 371 Date: Apr. 20, 2023

(30) Foreign Application Priority Data
Oct. 23, 2020 (EP) 20306263.3

(51) Int. Cl. G01N 33/574

(52) U.S. Cl. CPC G01N 33/57438; G01N 33/57484; G01N 2333/988

(57) ABSTRACT

A method of predicting the risk of recurrence in a subject undergoing treatment for, or having undergone treatment for, clear cell renal cell carcinoma (ccRCC), by comparing the level of extracellular vesicles, preferably microvesicles, expressing carbonic anhydrase 9 (CA9⁺ MVs) in a sample from the subject with a reference level. Also, a method of diagnosing ccRCC or identifying a risk of developing ccRCC, by comparing the level of CA9⁺ MVs in a sample from the subject with a reference level.

### FIELD OF INVENTION

The present invention relates to a method of predicting the risk of recurrence in a subject undergoing treatment for, or having undergone treatment for, clear cell renal cell carcinoma (ccRCC), by comparing the level of extracellular vesicles, preferably microvesicles, expressing carbonic anhydrase 9 (CA9⁺ MVs) in a sample from the subject with a reference level. Also described herein is a method of diagnosing ccRCC or identifying a risk of developing ccRCC, by comparing the level of CA9⁺ MVs in a sample from the subject with a reference level.

### BACKGROUND OF INVENTION

Renal cell carcinoma (RCC) currently represents the third most common urological cancer (Siegel et al., 2015. CA Cancer J Clin. 65(1):5-29), clear-cell renal cell carcinoma (ccRCC) being the most frequent RCC subtype. This cancer is difficult to detect and to treat. Currently, computed tomography or biopsy examinations are the main techniques used for the diagnosis and staging of tumor (Miller et al., 2019. CA Cancer J Clin. 69(5):363-385). The main therapeutic approach in early stage RCC, especially for ccRCC, is partial or radical nephrectomy, but disease recurrence occurs in 30-40% of patients. Tumor Node Metastasis (TNM) staging system and prognostic scores have been developed to estimate the risk of recurrence and the probability of survival after nephrectomy (Buti et al., 2012. Oncol Rev. 6(2):e18). But although diagnostic and prognostic techniques have increased the early detection of ccRCC and decreased the mortality rate (Vuyyala et al., 2019. Cureus. 11(8):e5531), almost half of the patients with ccRCC develop metastases after tumor resections, and indicators that predict the risk of metastasis are not available (Escudier et al., 2019. Ann Oncol. 30(5):706-720). For this raison, the discovery of prognostic markers that could predict the outcome of ccRCC after surgery is needed.

Carbonic anhydrase 9 (CA9), one of the most studied surface antigens in ccRCC, is to date the most promising biomarker to have these requirements. CA9 is a metalloenzyme weakly expressed in almost all tissues, exception made for certain gastrointestinal structures. However, CA9 is upregulated in hypoxic cells and is overexpressed in ccRCC, as a result of von Hippel-Lindau inactivation. Although RT-PCR is a preferred method to immunohistochemistry for the detection of CA9 in renal biopsy samples, blood-based assays may be the future for non-invasive diagnosis of renal tumors. Several studies have shown that, in ccRCC, values for serum and plasma soluble (s)-CA9 were higher than in other types of RCC. However, the correlation between CA9 expression and pathologic features provided controversial results. In fact, serum s-CA9 concentration determined by ELISA was shown to be correlated with T stage, Fuhrman grade and metastatic status. Conversely, in patients with localized disease, serum s-CA9 levels were correlated with tumor size but not with Fuhrman grade. In light of these results, it is unlikely that s-CA9 could be considered as a diagnostic tool. In line with this, quantitative RT-PCR for serum mRNA CA9 might be more specific and sensitive, but further studies are needed.

There remains thus a need for a robust, non-invasive diagnosis tool of renal tumors.

Extracellular vesicles (EVs) are a wide variety of small membrane-bound vesicles, including exosomes, microvesicles (MVs), and apoptotic bodies, released by almost all cell types, including tumor cells (Tkach & Théry, 2016. Cell. 164(6):1226-1232). They are also found in almost all types of body fluid, such as blood, urine, breast milk, saliva and semen (van Niel et al., 2018. Nat Rev Mol Cell Biol. 19(4):213-228). EVs contain nucleic acids, proteins and lipids, which are of particular interest for cancer biomarker research. Until now, many reports have shown that EV-associated RNAs are more stable than circulating RNAs and could be utilized for diagnostic approaches.

Although EVs may be considered as promising biomarkers in cancer, few EV biomarkers have been used into clinical practice. Several studies have shown that urinary EVs could serve as RCC biomarkers. Indeed, the expression levels of GSTA1, CEBPA and PCBD1 in EVs are decreased in the urine of ccRCC patients compared to RCC patients and healthy controls. Interestingly, one month after partial or radical nephrectomy, these levels increase to reach those of healthy subjects. In addition, proteomic analysis showed the efficacy of proteins, such as MMP-9, PODXL, DKK4, CA9 and ceruloplasmin, contained within urine EVs as biomarkers of RCC.

Here, the Inventors mainly focused on blood circulating MVs and established a novel method that will facilitate their clinical utilization. By flow cytometry, the Inventors have evaluated the levels of MVs expressing CA9 (CA9⁺ MVs) in plasma samples from RCC patients and healthy controls.

This method was more sensitive and rapid compared to conventional methods such as ELISA. Moreover, the Inventors have shown that, using this method, clinico-pathological variables could be defined with significant accuracy, such as the tumor size, this ISUP grade or the progression-free survival of patients following primary treatment (such as, e.g., surgery).

In brief, the Inventors have thus demonstrated that the detection of CA9 carried by circulating MVs represent diagnostic and prognostic biomarkers for RCC, especially for ccRCC.

### SUMMARY

The present invention relates to a method of predicting the risk of recurrence in a subject undergoing treatment for, or having undergone treatment for, clear cell renal cell carcinoma (ccRCC), comprising:
a) measuring the level of extracellular vesicles expressing carbonic anhydrase 9 (CA9⁺ EVs) in a sample previously obtained from the subject,
b) comparing the level of CA9⁺ EVs with a reference level,
c) assigning the subject to a high-risk group of ccRCC recurrence if the level of CA9⁺ EVs is substantially higher than the reference level, or assigning the subject to a low-risk group of ccRCC recurrence if the level of CA9⁺ EVs is substantially similar or lower than the reference level.

In one embodiment, the CA9⁺ EVs are microvesicles expressing carbonic anhydrase 9 (CA9⁺ MVs).

In one embodiment, the level of CA9⁺ EVs, preferably of CA9⁺ MVs, is expressed as an absolute number of CA9⁺ EVs, preferably of CA9⁺ MVs, in a given volume of sample.

In one embodiment, the absolute number of CA9⁺ EVs, preferably of CA9⁺ MVs, in a given volume of sample is determined by a method consisting of:
a) centrifuging the sample previously obtained from the subject at about 260 g for about 15 minutes,
b) centrifuging the supernatant retrieved after step a) at about 1500 g for about 20 minutes, and
c) measuring the absolute number of CA9⁺ EVs, preferably of CA9⁺ MVs, in a given volume of the supernatant retrieved after step b).

In one embodiment, the reference level is derived from the measurement of CA9⁺ EVs, preferably of CA9⁺ MVs, in a sample from a reference subject or in samples from a population of reference subjects, said reference subject(s) being known to have low risks of ccRCC recurrence.

In one embodiment, the reference level is about 350 CA9⁺ MVs/μL of sample.

In one embodiment, the sample is a blood sample.

In one embodiment, measuring the level of CA9⁺ EVs, preferably of CA9⁺ MVs, is carried out by flow cytometry.

The invention also relates to a method of diagnosing clear cell renal cell carcinoma (ccRCC) in a subject or of identifying a subject as being at risk of developing ccRCC, comprising:
a) measuring the level of extracellular vesicles expressing carbonic anhydrase 9 (CA9⁺ EVs) in a sample previously obtained from the subject,
b) comparing the level of CA9⁺ EVs with a reference level,
c) concluding that the subject is affected with, or is at risk of developing, ccRCC if the level of CA9⁺ EVs is substantially higher than the reference level.

In one embodiment, the CA9⁺ EVs are microvesicles expressing carbonic anhydrase 9 (CA9⁺ MVs).

In one embodiment, the level of CA9⁺ EVs, preferably of CA9⁺ MVs, is expressed as a percentage of CA9⁺ EVs, preferably of CA9⁺ MVs, out of the total extracellular vesicles, preferably microvesicles, in the sample.

In one embodiment, the reference level is derived from the measurement of CA9⁺ EVs, preferably of CA9⁺ MVs, in a reference subject or in a population of reference subjects not suffering from and/or not diagnosed with ccRCC.

In one embodiment, the reference level is 1.85% of CA9⁺ MVs out of the total microvesicles in the sample.

In one embodiment, diagnosing ccRCC consists of determining the tumor size and/or grading of the ccRCC.

In one embodiment, the sample is a blood sample.

In one embodiment, measuring the level of CA9⁺ EVs, preferably of CA9⁺ MVs, is carried out by flow cytometry.

### Definitions

In the present invention, the following terms have the following meanings:

“Carbonic anhydrase 9” or “CA9”, also termed “carbonate dehydratase IX”, “carbonic anhydrase IX”, are used interchangeably herein, and refer to a zinc metalloenzyme which belongs to a family of enzymes that are involved in reversible hydration of carbon dioxide to form bicarbonate and hydrogen ions. CA9 has limited expression in normal tissues, but is overexpressed in carcinoma cells lines. Expression is restricted to very few normal tissues (primarily in epithelial cells of gastric mucosa), the most abundant expression of CA9 being found in carcinoma cell lines. Human CA9 consists of an amino acid sequence with SEQ ID NO: 1 (Uniprot accession number Q16790-1, version 2; last modified on Dec. 6, 2005; Checksum: iBA67195483F0F5CE).

“Clear cell Renal Cell Carcinoma” and “ccRCC” are used interchangeably herein, and refer to the most common renal neoplasm seen in adults (70% of tumors derived from tubular epithelium). ccRCC can be as small as 1 cm or less and discovered incidentally, or it can be as bulky as several kilograms, and often presents pain, as a palpable mass or with hematuria, but a wide variety of paraneoplastic syndromes have been described. ccRCC might be clinically silent for years and may present with symptoms of metastasis. ccRCC has a characteristic gross appearance; the tumor is solid, lobulated, and yellow, with variegation due to necrosis and hemorrhage, with in some instances, the tumor circumscribed, or invade the perirenal fat or the renal vein.

“Diagnosis” refers to medical diagnosis, i.e., the process of identifying or determining a pathological state, disease or condition, such as RCC, preferably ccRCC.

“Extracellular vesicle” or “EV” refers to heterogenous vesicles formed by budding of the plasma membrane of eukaryotic cells, to the exterior of the cell. Microvesicles are secreted in larger amounts by cancer cells than normal cells. These membrane vesicles are heterogeneous in size with diameters ranging from about 10 nm to about 5000 nm, but also in biogenesis pathway or cellular source. All membrane vesicles shed by cells <0.8 μm in diameter are referred to herein collectively as “microvesicles”. Examples of extracellular vesicles include, but are not limited to, microvesicles, microvesicle-like particles, prostasomes, exosomes, dexosomes, texosomes, ectosomes, oncosomes, microparticles, apoptotic bodies, retrovirus-like particles, and human endogenous retrovirus (HERV) particles.

“Level” refers to the measured amount, quantity or concentration, whether relative or absolute, of a biomarker in a sample from a subject. The level of a biomarker can be determined relative to a control molecule in a sample, or relative to the level of the same biomarker in a reference population (i.e., relative to a reference level).

“Metastasis” refers to a process in which cancer cells travel from one organ or tissue to another non-adjacent organ or tissue. Cancer cells in the kidney can spread to tissues and organs of a subject, and conversely, cancer cells from other organs or tissue can invade or metastasize to the kidney. Cancerous cells from the kidney may invade or metastasize to any other organ or tissue of the body.

“Microvesicle” or “MV” refers to a type of extracellular vesicles, that are released into the extracellular environment by the outward budding and fission of the plasma membrane. They can be as small as 30 nm in diameter or as large as 1000 nm. Microvesicles play a role in intercellular communication and can transport molecules such as mRNA, miRNA, and proteins between cells.

“Prognosis” refers to the likelihood of cancer-attributable death or cancer progression, including recurrence and metastatic spread of a neoplastic disease, during the natural history of the disease, or to the likelihood of a beneficial outcome whether following a specific treatment or not, wherein a beneficial response means an improvement in any measure of patient status including, but not limited to, overall survival, long-term survival (i.e., survival for at least 3, preferably at least 5, 8, or 10 years following diagnosis, surgery or other treatment), recurrence-free survival, and distant recurrence-free survival. Accordingly, a “good prognosis” or “positive prognosis” refers to a beneficial clinical outcome such as long-term survival without recurrence; and a “bad prognosis” or “negative prognosis” refers to a negative clinical outcome such as cancer recurrence.

“Reference level” refers to the level of a biomarker in a sample from a reference subject or to the mean or median level of a biomarkers in samples from several subjects in a reference population. A reference level can be a normal reference level or a disease-state reference level. A normal reference level is the level of a biomarker in a substantially healthy subject or in several substantially healthy subjects in a reference population, such as a subject (or several subjects) who is/are not suffering from or otherwise was/were not diagnosed with, RCC, preferably ccRCC. A disease-state reference level is the level of a biomarker in a diseased subject or in several diseased subjects in a reference population, such as a subject (or several subjects) who is/are suffering from or otherwise was/were diagnosed with, RCC, preferably ccRCC.

“Recurrence” refers to local or distant recurrence (i.e., metastasis) of cancer. For example, RCC can recur locally, in the case of partial nephrectomy, radiofrequency ablation or cryoablation. The cancer may also affect the surrounding lymph nodes, the ipsilateral adrenal gland, the perirenal fatty tissue, the renal fossa, or the psoas muscle. Renal cell carcinoma can also spread to other organs such as the lung, the bone, the liver. Recurrence is typically determined by, e.g., imaging study or biopsy.

“Renal Cell Carcinoma” and “RCC” are used interchangeably herein, and refer to a tumor of the kidney. Tumors of the kidney can be malignant or benign and are the most common primary malignant kidney tumor. RCC usually begins in the cells that line the small tubes of each nephron. Renal cell tumors can grow as a single mass, and multiple RCC tumors can develop on a single kidney or both kidneys. The term RCC encompasses different subtypes of RCC. In a microscopic context, there are four major histologic subtypes of renal cell cancer: clear cell (conventional RCC, 75%), papillary (15%), chromophobic (5%) and collecting duct (2%). The 2016 World Health Organization (WHO) classification of genitourinary tumours (WHO Classification of Tumours Editorial Board, 2016. WHO classification of tumours of the urinary system and male genital organs (4^{th} ed., Vol. 8, WHO Classification of Tumours) (Moch et al., Ed.). Lyon, France: IARC Press; Moch et al., 2016. Eur Urol. 70(1):93-105) recognizes tens of subtypes of renal neoplasms.

“Risk classification” means a grouping of subjects by the level of risk (or likelihood) that the subject will experience a particular clinical outcome. A subject may be classified into a risk group or classified at a level of risk based on the methods of the present disclosure, e.g., high or low risk. A “risk group” is a group of subjects with a similar level of risk for a particular clinical outcome.

“Sample” refers to any biological material obtained via suitable methods known to the person skilled in the art from a subject. The sample may be collected in a clinically acceptable manner, e.g., in a way that cells, nucleic acids (such as DNA and RNA), proteins and/or extracellular vesicles are preserved. A “sample” may be a body tissue and/or a bodily fluid, preferably a bodily fluid. Examples of bodily fluids include, but are not limited to, blood, plasma, serum, lymph, ascetic fluid, cystic fluid, urine, bile, nipple exudate, vomitus, breast milk, tears, wound drainage, feces, vaginal secretions, synovial fluid, bronchoalveolar lavage fluid, sputum, amniotic fluid, peritoneal fluid, cerebrospinal fluid, pleural fluid, pericardial fluid, semen, saliva, sweat and alveolar macrophages. In one embodiment of the invention, a “sample” may be a blood sample (including whole blood, plasma and serum).

“Subject” refers to an animal, preferably a mammal, more preferably a human. In one embodiment, the subject is a patient, i.e. a recipient of health care services. Preferably, the subject is a cancer patient, i.e. he/she was previously diagnosed with cancer.

“Substantially healthy”, with reference to a subject or a population of subjects, means that said subject (or subjects in the population) is/are not suffering from or otherwise was/were not diagnosed with, RCC, preferably with ccRCC.

“Surgery” applies to surgical methods undertaken for removal of cancerous tissue, including open surgery partial or radical nephrectomy, radiofrequency ablation (RFA), cryoablation (CRA), excision, dissection and tumor biopsy/removal.

### DETAILED DESCRIPTION

The present invention relates to a method of diagnosing a renal cell carcinoma in a subject. Another object of the invention is a method of identifying a subject as being at risk of developing a renal cell carcinoma. Another object of the invention is a method of predicting the risk of recurrence—or the chances of recurrence-free survival—in a subject undergoing treatment for or having undergone treatment for renal cell carcinoma.

In one embodiment of the methods, the renal cell carcinoma is clear cell renal cell carcinoma (ccRCC).

In one embodiment, the methods according to the present invention comprise a step of providing a sample from a subject.

In one embodiment, the sample is a bodily fluid. Examples of bodily fluids include, but are not limited to, blood, plasma, serum, lymph, ascetic fluid, cystic fluid, urine, bile, nipple exudate, vomitus, breast milk, tears, wound drainage, feces, vaginal secretions, synovial fluid, bronchoalveolar lavage fluid, sputum, amniotic fluid, peritoneal fluid, cerebrospinal fluid, pleural fluid, pericardial fluid, semen, saliva, sweat and alveolar macrophages.

In one embodiment, the sample is a blood sample (including whole blood, plasma and serum). In one embodiment, the sample is a whole blood sample. In one embodiment, the sample is a plasma sample.

In one embodiment, the sample was previously taken from the subject, i.e., the methods of the invention do not comprise an active step of recovering a sample from the subject. Consequently, according to this embodiment, the methods of the invention are non-invasive methods, i.e., the methods of the invention are in vitro methods.

In one embodiment, the sample was previously taken from the subject into a suitable container, such as, e.g., in an EDTA tube.

In one embodiment, the methods according to the present invention comprise a step of processing the sample.

In one embodiment, the step of processing the sample comprises centrifuging a whole blood sample in order to obtain platelet-rich plasma. In one embodiment, platelet-rich plasma may be obtained by centrifugation at about 260 g for about 15 minutes.

In one embodiment, the step of processing the sample comprises centrifuging a whole blood sample in order to obtain platelet-free plasma. In one embodiment, the step of processing the sample comprises centrifuging a platelet-rich plasma sample in order to obtain platelet-free plasma. In one embodiment, platelet-free plasma may be obtained by centrifugation at about 1500 g for about 20 minutes.

In one embodiment, the methods according to the present invention comprise a step of measuring the level of extracellular vesicles (EVs) expressing carbonic anhydrase 9 (CA9) (CA9⁺ EVs) in a sample from the subject, preferably the level of microvesicles (MVs) expressing carbonic anhydrase 9 (CA9) (CA9⁺ MVs) in a sample from the subject.

In one embodiment, the level of CA9⁺ EVs, preferably of CA9⁺ MVs, may be measured by any conventional method known to the one skilled in the art, including, but not limited to, flow cytometry using antibodies directed against CA9; ELISA using antibodies directed against CA9; mass spectrometry; and the like.

In one embodiment, the level of CA9⁺ EVs, preferably of CA9⁺ MVs, is measured by flow cytometry using antibodies directed against CA9. Several antibodies directed against human CA9 useful for flow cytometry applications are commercially available, such as REA658 (Miltenyi Biotec ref. #130-110-057), SP106 (AbCam ref. #ab105226), 2D3 (AbCam ref. #ab107257), CA9/781 (AbCam ref. #ab216021), 10F7A8 (AbCam ref. #ab181464), MM0610-3B15 (AbCam ref. #ab135159), 053 (AbCam ref. #ab275578), GT12 (ThermoFisher ref #MA5-16318), 66.4.C2 (LifeSpan BioSciences ref. #LS-C357729-20), OTI1G7 (OriGene ref. #TA500623S), SPM314 (OriGene ref. #AM50188PU), SPM487 (OriGene ref. #AM33258PU), girentuximab (Creative Biolabs), to name but a few.

In one embodiment, the level of CA9⁺ EVs, preferably of CA9⁺ MVs, may be measured as a percentage of the total microvesicles in the sample.

In one embodiment, the level of CA9⁺ EVs, preferably of CA9⁺ MVs, may be measured as an absolute value, i.e., an absolute number of CA9⁺ EVs, preferably of CA9⁺ MVs, in a given volume of sample (e.g., per μL of plasma).

In one embodiment, the methods according to the present invention comprise a step of preparing a hard and/or soft copy comprising the value of the level of measured CA9⁺ EVs, preferably of CA9⁺ MVs.

Examples of hard copies include, but are not limited to, print-outs, hand-written information, photographs, data as originally obtained (such as, e.g., from a flow cytometer).

Examples of soft copies include, but are not limited to, any form of computer readable files such as, e.g., the originally obtained data output from the machine performing the measurements (e.g., a flow cytometer) or from the respective analysis program; word or other text software documents containing the values; screen shots.

It will be appreciated that the values comprised in said hard or soft copy can be, e.g., raw values (i.e., original data as obtained) or calculated values (e.g., in the form of numerical values derived from the measurements).

In one embodiment, the methods of the present invention comprise a step of comparing the level of CA9⁺ EVs, preferably of CA9⁺ MVs, with a reference level.

In one embodiment, the reference level is derived from the measurement of CA9⁺ EVs, preferably of CA9⁺ MVs, as described above, in a reference subject.

In one embodiment, the reference level is derived from the measurement of CA9⁺ EVs, preferably of CA9⁺ MVs, as described above, in a reference population.

As used herein, a “reference population” is a population comprising at least 2, preferably at least 5, at least 10, at least 15, at least 20, at least 25, at least 30, at least 35, at least 40, at least 45, at least 50, at least 75, at least 100 or more reference subjects.

In one embodiment of the methods according to the invention, the reference subject(s) is/are (a) substantially healthy subject(s).

In one embodiment, comparing the level of CA9⁺ EVs, preferably of CA9⁺ MVs, with a reference level may include a comparison “by eye”.

In one embodiment, comparing the level of CA9⁺ EVs, preferably of CA9⁺ MVs, with a reference level may include one or more forms of statistical analysis.

Examples of such statistical analysis include, but are not limited to, regression analysis, univariate analysis, multivariate analysis, variation calculations, best-fit analysis, curve fitting, extrapolation, interpolation, least squares, mean calculations, simulation analysis, logrank test, Kaplan-Meier estimator, and the like.

In one embodiment, comparing the level of CA9⁺ EVs, preferably of CA9⁺ MVs, with a reference level may comprise or consist of comparing a statistical or mathematical representation (such as, e.g., absolute values, mean, median, or regression) of the level of CA9⁺ EVs, preferably of CA9⁺ MVs, with a statistical or mathematical representation (such as, e.g., absolute values, mean, median, or regression) of the reference level.

In one embodiment, the difference between the level of CA9⁺ EVs, preferably of CA9⁺ MVs, and the reference level is different with statistical significance.

By “different with statistical significance”, it is meant that, in a statistical analysis, the difference between the level of CA9⁺ EVs, preferably of CA9⁺ MVs, and the reference level shows a p value at or below 0.05, 0.04, 0.03, 0.02, 0.01, or less.

In one embodiment, appreciation of the difference between the level of CA9⁺ EVs, preferably of CA9⁺ MVs, and the reference level is left to the physician who is able to interpret a level of CA9⁺ EVs, preferably of CA9⁺ MVs, in comparison with a reference level, in particular the light of the examples disclosed herein.

In one embodiment, the methods of the present invention comprise a step of diagnosing the subject as being affected with or as being at risk of developing a RCC (preferably ccRCC).

In one embodiment, the subject is diagnosed as being affected with or as being at risk of developing a RCC (preferably ccRCC) if the level of CA9⁺ EVs, preferably of CA9⁺ MVs, is substantially higher than the reference level.

The term “substantially higher” denotes a sufficiently high degree of difference between the level of CA9⁺ EVs, preferably of CA9⁺ MVs, and the reference level, such as, different with statistical significance.

In one embodiment, the level of CA9⁺ EVs and the reference level are expressed as a percentage of the total extracellular vesicles.

In one embodiment, the level of CA9⁺ MVs and the reference level are expressed as a percentage of the total microvesicles.

In one embodiment, the subject is diagnosed as being affected with or as being at risk of developing a RCC (preferably ccRCC) if the percentage of CA9⁺ EVs, preferably of CA9⁺ MVs, out of the total microvesicles in a sample from the subject is substantially higher than the percentage of CA9⁺ EVs, preferably of CA9⁺ MVs, out of the total microvesicles in a sample from a reference subject or from samples from a reference population.

In one embodiment, the reference level of CA9⁺ EVs is at least 1% of CA9⁺ EVs out of the total extracellular vesicles in the sample, such as 1%±0.5%, 1.5%±0.5%, 2%±0.5%, 2.5%±0.5%, 3%±0.5% or more of CA9⁺ EVs out of the total extracellular vesicles; preferably the reference level of CA9⁺ EVs is about 1.85% of CA9⁺ EVs out of the total extracellular vesicles in the sample.

In one embodiment, the reference level of CA9⁺ MVs is at least 1% of CA9⁺ MVs out of the total microvesicles in the sample, such as 1%±0.5%, 1.5%±0.5%, 2%±0.5%, 2.5%±0.5%, 3%±0.5% or more of CA9⁺ MVs out of the total microvesicles; preferably the reference level of CA9⁺ MVs is about 1.85% of CA9⁺ MVs out of the total microvesicles in the sample.

In one embodiment, the step of diagnosing comprises determining the size of the RCC (preferably the ccRCC). In one embodiment, the step of diagnosing comprises grading the RCC (preferably the ccRCC).

In one embodiment, the level of CA9⁺ EVs, preferably of CA9⁺ MVs, is positively correlated with the tumor size.

In one embodiment, the level of CA9⁺ EVs, preferably of CA9⁺ MVs, is positively correlated with the ccRCC grade. In one embodiment, the Fuhrman grading system or the ISUP grading system is used to determine the ccRCC grade, preferably, the ISUP grading system is used to determine the ccRCC grade.

The Fuhrman grading system (Fuhrman et al., 1982. Am J Surg Pathol. 6(7):655-663) has been extensively used by pathologists in Europe and the United States (Zisman et al., 2001. J Clin Oncol. 19(6):1649-1657; Zisman et al., 2002. J Clin Oncol. 20(23):4559-4566; Frank et al., 2002. J Urol. 168(6):2395-2400; Karakiewicz et al., 2007. J Clin Oncol. 25(11):1316-1322); this system categorizes RCC with grades 1, 2, 3, and 4 based on nuclear characteristics and has represented one of the most significant prognostic variables in patients with all stages of RCC. Another grading system has been proposed by the International Society of Urologic Pathologists (ISUP) (Delahunt et al., 2014. Eur Urol. 66(5):795-798). The ISUP grading system categorizes RCC with grades I, II, III and IV, as follows:
Grade I: tumor cell nucleoli invisible or small and basophilic at 400× magnification;
Grade II: tumor cell nucleoli conspicuous at 400× magnification but inconspicuous at 100× magnification;
Grade III: tumor cell nucleoli eosinophilic and clearly visible at 100× magnification;
Grade IV: tumors showing extreme nuclear pleomorphism and/or containing tumor giant cells and/or the presence of any proportion of tumor showing sarcomatoid and/or rhabdoid dedifferentiation.

In one embodiment, the methods of the present invention comprise a step of prognosing a risk of recurrence—or the chances of recurrence-free survival—in the subject undergoing or having undergone treatment for RCC (preferably for ccRCC).

In one embodiment, the subject is assigned to a high-risk group of RCC (preferably of ccRCC) recurrence if the level of CA9⁺ EVs, preferably of CA9⁺ MVs, is substantially higher than the reference level.

According to this embodiment, the reference level is derived from the measurement of CA9⁺ EVs, preferably of CA9⁺ MVs, as described above, in a reference subject or in a reference population, wherein said reference subject is a subject known to have low risks of RCC (preferably of ccRCC) recurrence.

Alternatively, the subject is assigned to a high-risk group of RCC (preferably of ccRCC) recurrence if the level of CA9⁺ EVs, preferably of CA9⁺ MVs, is substantially similar or higher than the reference level.

According to this embodiment, the reference level is derived from the measurement of CA9⁺ EVs, preferably of CA9⁺ MVs, as described above, in a reference subject or in a reference population, wherein said reference subject is a subject known to have high risks of RCC (preferably of ccRCC) recurrence.

In one embodiment, the subject is assigned to a low-risk group of RCC (preferably of ccRCC) recurrence if the level of CA9⁺ EVs, preferably of CA9⁺ MVs, is substantially lower than the reference level.

According to this embodiment, the reference level is derived from the measurement of CA9⁺ EVs, preferably of CA9⁺ MVs, as described above, in a reference subject or in a reference population, wherein said reference subject is a subject known to have high risks of RCC (preferably of ccRCC) recurrence.

Alternatively, the subject is assigned to a low-risk group of RCC (preferably of ccRCC) recurrence if the level of CA9⁺ EVs, preferably of CA9⁺ MVs, is substantially similar or lower than the reference level.

According to this embodiment, the reference level is derived from the measurement of CA9⁺ EVs, preferably of CA9⁺ MVs, as described above, in a reference subject or in a reference population, wherein said reference subject is a subject known to have low risks of RCC (preferably of ccRCC) recurrence.

In one embodiment, the level of CA9⁺ EVs, preferably of CA9⁺ MVs, and the reference level are expressed as an absolute value, i.e., an absolute number of CA9⁺ EVs, preferably of CA9⁺ MVs, in a given volume of sample (e.g., per p L of plasma).

In one embodiment, the absolute number of CA9⁺ EVs, preferably of CA9⁺ MVs, in a given volume of sample may be measured by a method comprising or consisting of:
a) centrifuging the sample at about 260 g for about 15 minutes,
b) centrifuging the supernatant retrieved after step a) at about 1500 g for about 20 minutes, and
c) measuring the level of CA9⁺ EVs, preferably of CA9⁺ MVs, in a given volume of the supernatant retrieved after step b), such as, e.g., by flow cytometry.

In one embodiment, the reference level of CA9⁺ EVs is at least 200 CA9⁺ EVs/μL of plasma, such as 200, 250, 300, 350, 400 or more CA9⁺ EVs/μL of plasma; preferably the reference level of CA9⁺ MVs is about 350 CA9⁺ EVs/μL of plasma.

In one embodiment, the reference level of CA9⁺ MVs is at least 200 CA9⁺ MVs/μL of plasma, such as 200, 250, 300, 350, 400 or more CA9⁺ MVs/μL of plasma; preferably the reference level of CA9⁺ MVs is about 350 CA9⁺ MVs/μL of plasma.

It will be readily understood by the one skilled in the art that the level of CA9⁺ EVs, preferably of CA9⁺ MVs, may vary depending on how the sample is processed. Any methods to measure the level of CA9⁺ EVs, preferably of CA9⁺ MVs, and any associated reference levels described herein, are given for exemplary purposes; however, one skilled in the art will understand that both the level of CA9⁺ EVs, preferably of CA9⁺ MVs, and the reference level shall be measured using the same method to have comparable data.

Another object of the invention is a method of treating renal cell carcinoma (RCC) in a subject in need thereof.

In one embodiment of the method, the renal cell carcinoma is clear cell renal cell carcinoma (ccRCC).

In one embodiment, the method according to the invention comprises a step of diagnosing RCC (preferably ccRCC) in a subject, according to the methods described herein. In one embodiment, the method according to the invention comprises a step of identifying a subject as being at risk of developing RCC (preferably ccRCC), according to the methods described herein.

In one embodiment, where the subject has been diagnosed with, or is at risk of developing, RCC (preferably ccRCC), the method according to the invention comprises a step of treating the subject for RCC (preferably for ccRCC).

Means and methods to treat a subject for RCC (preferably ccRCC) are known to the one skilled in the art. These include, but are not limited to, surgery and immunotherapy. Surgery aims at removing the cancer and optionally, part of the kidney surrounding it. In early stage RCC (preferably ccRCC), radiofrequency ablation (RFA), cryoablation (CRA), or partial nephrectomy (PN) by open surgery can be carried out to remove part of the kidney with the cancer. If RCC (preferably ccRCC) is in the middle of the kidney, or if the tumor is large, the entire kidney might have to be removed by open surgery radical nephrectomy (RN). Immunotherapy aims at targeting phenotypic changes in cancer cells to specifically stop their growth and spread. Examples of immunotherapies used for treating RCC (preferably ccRCC) include, but are not limited to, cabozantinib, axitinib, sunitinib, sorafenib, and pazopanib.

In one embodiment, the method according to the invention comprises a step of predicting the risk of recurrence—or the chances of recurrence-free survival—in a subject undergoing treatment for or having undergone treatment for RCC (preferably ccRCC), according to the methods described herein.

In one embodiment, where the subject has been assigned to a high-risk of RCC (preferably ccRCC) recurrence group, the method according to the invention comprises a step of treating the subject for recurrent RCC (preferably for recurrent ccRCC). The subject may be further treated depending on the primary treatment. For example, after radiofrequency ablation (RFA), cryoablation (CRA), or partial nephrectomy (PN) by open surgery, the subject may be further treated by RFA, CRA or PN, if possible, or by open surgery radical nephrectomy (RN). If the subject has already undergone open surgery radical nephrectomy (RN) as primary treatment, said subject may be further treated by surgical excision or by immunotherapy.

In one embodiment, where the subject has been assigned to a low-risk of RCC (preferably ccRCC) recurrence group, the method according to the invention comprises a step of placing the subject under active surveillance.

By “active surveillance” or “watchful waiting”, it is meant closely monitoring a subject's condition without giving any treatment until symptoms appear or change.

In one embodiment, the subject is an animal, preferably a mammal, more preferably a primate. In one embodiment, the subject is a human.

In one embodiment, the subject is a male. In one embodiment, the subject is female.

In one embodiment, the subject is a child, an adolescent or an adult.

In one embodiment, the subject is above the age of 40 years. In one embodiment, the subject is above the age of 50 years. In one embodiment, the subject is above the age of 60 years. In one embodiment, the subject is above the age of 70 years. In one embodiment, the subject is above the age of 80 years or more.

In one embodiment, the subject is aged from 0 to 20 years old. In one embodiment, the subject is aged from 20 to 40 years old. In one embodiment, the subject is aged from 40 to 50 years old. In one embodiment, the subject is aged from 50 to 55 years old. In one embodiment, the subject is aged from 55 to 60 years old. In one embodiment, the subject is aged from 60 to 65 years old. In one embodiment, the subject is aged from 65 to 70 years old. In one embodiment, the subject is aged from 70 to 75 years old. In one embodiment, the subject is aged from 75 to 80 years old. In one embodiment, the subject is aged from 80 to 85 years old or more.

In one embodiment, the subject is/was not diagnosed with RCC (preferably with ccRCC). In one embodiment, the subject is at risk of being diagnosed with RCC (preferably with ccRCC). In one embodiment, the subject is/was diagnosed with RCC (preferably with ccRCC).

In one embodiment, a subject is considered as being at risk of being diagnosed with RCC (preferably with ccRCC), if said subject displays at least one, at least two, at least three or more of the following symptoms: a lump in the abdomen, hematuria, unexplained weight loss, loss of appetite, fatigue, vision problems, persistent pain in the side, and/or excessive hair growth in women.

In one embodiment, the subject was not previously treated for RCC (preferably for ccRCC). In one embodiment, the subject was previously treated for RCC (preferably for ccRCC). In one embodiment, the subject is currently treated for RCC (preferably for ccRCC).

Examples of RCC treatments include, but are not limited to, surgery and immunotherapy. Surgery aims at removing the cancer and optionally, part of the kidney surrounding it. In early stage RCC (preferably ccRCC), radiofrequency ablation (RFA), cryoablation (CRA), or partial nephrectomy (PN) by open surgery can be carried out to remove part of the kidney with the cancer. If RCC (preferably ccRCC) is in the middle of the kidney, or if the tumor is large, the entire kidney might have to be removed by open surgery radical nephrectomy (RN). Immunotherapy aims at targeting phenotypic changes in cancer cells to specifically stop their growth and spread. Examples of immunotherapies used for treating RCC (preferably ccRCC) include, but are not limited to, cabozantinib, axitinib, sunitinib, sorafenib, and pazopanib.

### BRIEF DESCRIPTION OF THE DRAWINGS

FIGS. 1A-E are a set of flow cytometry plots and graphs showing the detection by flow cytometry of circulating MVs by staining for the cell surface marker CA9.

FIG. 1A: Representative flow cytometry plot for CA9-MVs from one control and one RCC patient after staining with an anti-CA9 antibody.

FIG. 1B: Graph showing the mean of the percentage of CA9⁺ MVs/μL of plasma from controls (n=16) and RCC patients (n=76). Data are shown as mean values±SEM. *P<0.05.

FIG. 1C: Representative flow cytometry plot for CA9-MVs from one control, one n-ccRCC patient and one ccRCC patient. The percentages show the number of positive events for staining of plasma circulating MVs visualized by plotting CA9 marker (x axis) vs FSlog properties (y axis) and gated based on isotype control.

FIG. 1D: Graph showing the mean of percentage of CA9⁺ MVs/μL of plasma in controls (n=16), n-ccRCC patients (n=12) and ccRCC (n=64) patients. Data are shown as mean values±SEM. *P<0.05.

FIG. 1E: Graph showing the spearman correlation between the percentage of MVs expressing CA9/μl of plasma detected by flow cytometry and the tumor size (cm).

FIGS. 2A-B are a set of flow cytometry plots and graph showing the comparison of the percentage of CA9⁺ MVs from plasma of ccRCC patients before and 1 month after surgical removal of tumor.

FIG. 2A: Representative flow cytometry plots for CA9⁺ MVs from one RCC patient before (day 0) and 1 month after (+1 month) nephrectomy.

FIG. 2B: Graph showing the mean of the percentage of CA9⁺ MVs from RCC patients before (ccRCC) and 1 month after (ccRCC 1M) nephrectomy (n=10). Wilcoxon test was used to determine statistical significance. *P<0.05.

FIGS. 3A-C are a set of graphs showing the concentration of s-CA9 in the serum in controls, RCC patients, n-ccRCC patients and ccRCC patients measured by ELISA.

FIG. 3A: Graph showing the mean of plasma s-CA9 concentration expressed in pg/mL in controls (n=16) and RCC patients (n=76). Data are shown as mean values±SEM. **P<0.01.

FIG. 3B: Graph showing the mean of plasma s-CA9 concentration expressed in pg/mL in controls (n=16), n-ccRCC patients (n=12) and ccRCC (n=64) patients. Data are shown as mean values±SEM. **P<0.01.

FIG. 3C: Graph showing the spearman correlation between plasma concentration of s-CA9 (pg/mL) observed by ELISA and tumor size (cm).

FIG. 4 is a receiver operating characteristic (ROC) curve analysis using the percentage of CA9⁺ MVs detected by flow cytometry.

FIGS. 5A-B are a set of graphs showing the progression free survival of all patients according to the number of circulating CA9⁺ MVs detected by flow cytometry (A) or s-CA9 concentration detected by ELISA (B).

FIG. 5A: RCC patients with low absolute number of CA9⁺ MVs (<350) measured by flow cytometry revealed a better progression-free survival than those with high value (≥350).

FIG. 5B: No correlation was observed between s-CA9 concentration measured by ELISA and the progression-free survival in these patients.

### EXAMPLES

The present invention is further illustrated by the following examples.

### Example 1: Detection of Microvesicles Carrying CA9 by Flow Cytometry

### Materials and Methods

### Patients Included in the Cohort

The clinical data reported for this study were collected within the framework of the UroCCR project (NCT03293563), CNIL authorization number DR-2013-206. This prospective monocentric study included all patients treated surgically for a localized renal tumor between May 2017-January 2019.

Pre-operative clinical data were collected and anonymized via a national renal cancer database (Réseau Français de Recherche sur le Cancer du Rein—Uro-CCR). They included age, sex and tumor size. Tumors were classified according to the TNM 2009 classification (Kidney (ICD-O C64), 2010. In Sobin et al. (Eds.), TNM classification of malignant tumours (7^{th} Ed., pp. 255-257). Oxford, UK: Wiley-Blackwell), histological subtypes were recorded according to the 2015 WHO classification of kidney tumors (Moch et al., 2016. Eur Urol. 70(1):93-105). ISUP grade for renal cell carcinomas (Delahunt et al., 2019. Histopathology. 74(1):4-17) was analyzed. The date of nephrectomy was considered as the start of follow-up.

In total, 16 individuals served as controls; 8 of these had been admitted in hospital for urological conditions other than RCC (infections, urinary stones, etc.). Other plasma controls were obtained from 8 healthy blood donors.

77 patients (52 male and 25 female) were included in the study. A patient with a carcinoma different from RCC was excluded. Among RCC patients, 71 had a localized or locally advanced renal cancer and 6 had a metastatic renal carcinoma. Patient characteristics are summarized in Table 1. At the end of the follow-up, 9 patients died with 5 deaths from cancer. Median follow-up for patient without metastasis was 13.5 (3-48) months. Among patients without metastasis at diagnosis, 11 experienced a local or a metastatic recurrence.

Lastly, the blood samples from 10 patients were collected 1 month after the tumor resection, and these samples were used to determine whether the levels of CA9⁺ MVs in blood were changed after the tumor resection.

TABLE 1
Patient and tumor characteristics

| Characteristics | | Controls | n-ccRCC | ccRCC |
|---|---|---|---|---|
| Age at | Median (IQR) | 40 (24-66) | 72 (52-80) | 65 (38-85) |
| diagnosis (years) | | | | |
| Gender | Male | 10 | 9 | 42 |
| | Female | 6 | 3 | 22 |
| TNM stage | T1-T2 | — | 10 | 32 |
| | T3-T4 | — | 1 | 32 |
| ISUP grade | I-II | — | 6 | 32 |
| | III-IV | — | 2 | 32 |
| Size | ≤4 cm | — | 4 | 27 |
| | >4 cm | — | 8 | 37 |
| Recurrence | | — | 2 | 13 |
| Cancer-related death | | — | 1 | 4 |

ccRCC, clear cell renal cell carcinoma; n-ccRCC, non-clear cell renal cell carcinoma (i.e., other types of renal cell carcinoma); IQR: interquartile range; TNM: (T) tumor, (N) node, (M) metastasis; ISUP: International society of urological pathology.

### Sample Processing

Peripheral blood (8 mL) was collected in EDTA-treated tubes (Vacutainers, Becton Dickinson, Le Pont de Claix, France) from a peripheral vein using a 21-gauge needle to minimize platelet activation and was processed for assay within 2 hours from collection (Agouni et al., 2008. Am J Pathol. 173(4):1210-1219). Blood collection was carried out before surgery.

Briefly, blood was centrifuged at 260 g for 15 minutes and platelet-rich plasma was separated from whole blood. Then, platelet-rich plasma was further centrifuged at 1500 g for 20 minutes to obtain platelet-free plasma (PFP). PFP were frozen and stored at −80° C. until subsequent use.

### Characterization of Microvesicles Harboring CA9

Characterization of plasma MVs was performed by flow cytometry using a specific antibody against Carbonic Anhydrase 9 (CA9)-PE, (Cat #130-110-057, Miltenyi Biotec, Bergisch Gladbach, Germany). The antibody was incubated for 30 minutes at 4° C. Irrelevant human IgG were used as isotype-matched negative control. Samples were analyzed in a flow cytometer 500 MPL system (Beckman Coulter, Villepinte, France). MV quantification was performed using calibrated 10 μm-sized Flowcount beads (Beckman Coulter) of known concentration on FC500 cytometer (Beckman Coulter, France).

### Results

To determine whether MV quantification can be used as a diagnostic tool in clinical situation, we analyzed circulating MVs in the plasma without a purification step of MVs. The optimal configuration and settings for quantitative and qualitative flow cytometry analyses of MVs have been adapted from the study by Nolte-'T Hoen et al. (2013. J Leukoc Biol. 93(3):395-402). Therefore, using the optimal settings, 100 nm fluorescent polystyrene beads were efficiently detected above background noise.

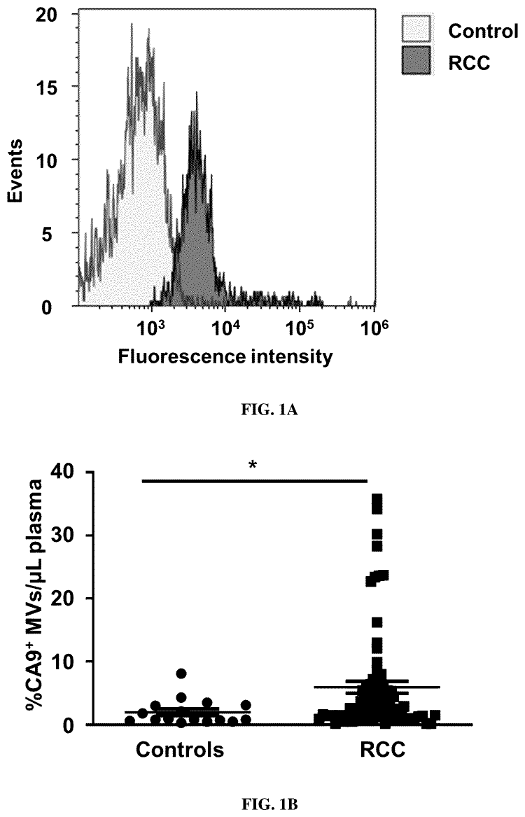

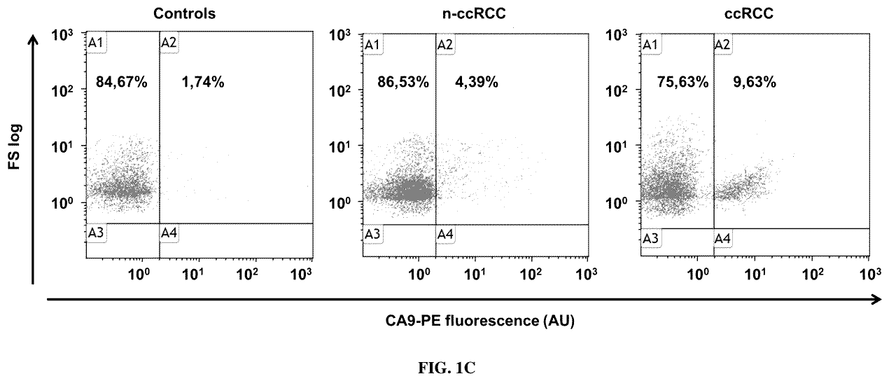

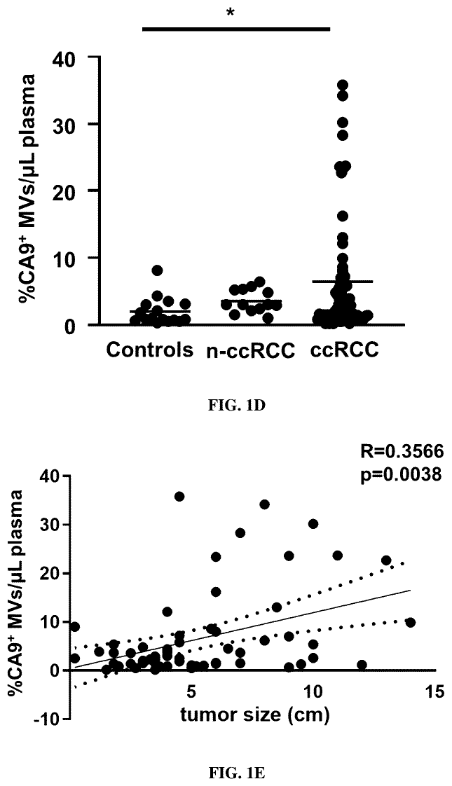

CA9 was detected as circulating MV cargo component by flow cytometry. RCC patient samples exhibited a strong positive staining by anti-CA9 antibody, whereas the corresponding control sample showed only a weak fluorescence signal (FIG. 1A).

We assessed whether the percentage of MVs carrying CA9 (CA9⁺ MVs) in the plasma was associated with disease, stage or grade. Firstly, we found no significant association between levels of CA9 carried by circulating MVs and gender, age, or ccRCC TNM stage (Table 2). Next, we found that CA9⁺ MVs percentage was significantly higher in plasma from RCC patients than in plasma from controls (FIG. 1B). In subgroups of RCC, CA9⁺ MVs were significantly higher in plasma from ccRCC, when compared with healthy controls (FIGS. 1C and 1D). Moreover, the level of CA9⁺ MVs in ccRCC patients was markedly more elevated than in n-ccRCC patient samples (FIGS. 1C and 1D).

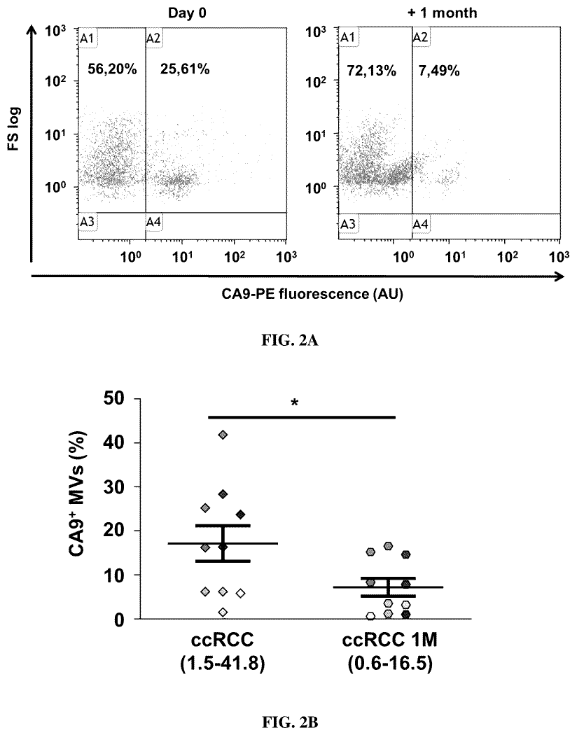

Interestingly, the percentage of CA9⁺ MVs in the plasma from ccRCC patients decreased after surgery (FIGS. 2A and 2B), suggesting that the signal of CA9 obtained by flow cytometry originates from tumor-derived MVs.

Moreover, in ccRCC patients, CA9⁺ MVs correlated with tumor size (FIG. 1E) and with ISUP grade I-II versus III-IV (Table 2).

TABLE 2
Relationship between clinical characteristics and levels of
circulating CA9⁺ MVs (in % of the total circulating MVs).

| | |
|---|---|
| ccRCC | CA9⁺ MVs |

| Parameter | patients (n) | Mean | p-value |
|---|---|---|---|
| Gender | | | 0.36 |
| Male | 42 | 7.3 | |
| Female | 22 | 4.09 | |
| Age | | | 0.72 |
| ≥60 | 39 | 6.13 | |
| <60 | 25 | 6.93 | |
| TNM Stage | | | 0.65 |
| T1-T2 | 32 | 5.11 | |
| T3-T4 | 32 | 7.78 | |
| ISUP grade | | | 0.05 |
| I-II | 32 | 4.3 | |
| III-IV | 32 | 8.56 | |

ccRCC: clear cell renal cell carcinoma;
TNM: (T) tumor, (N) node, (M) metastasis;
ISUP: International society of urological pathology

### Example 2: Quantitative Analysis of Plasma CA9 Concentration

### Materials and Methods

### Measure by ELISA of CA9 Concentration in the Plasma

Soluble plasma CA9 (s-CA9) was quantified by human carbonic anhydrase 9 Quantikin ELISA kit (Cat #DCA900, R&D Systems, Minneapolis, MN, USA) according to the protocol of the manufacturer.

Briefly, plasma samples or standard control samples were incubated on microplates coated with a specific antibody to CA9 for 2 hours at room temperature. The plates were washed to remove unbound antibodies.

After incubation of a conjugate solution, a substrate solution was added. Color development was stopped after 30 minutes. A microplate reader (VICTOR Multilabel plate reader) was used to determine colorimetric densities at 450 nm.

Final results were calculated according to the standard curve. Results were expressed in pg/mL. The mean limit of quantification of this method was 2.28 pg/mL.

### Results

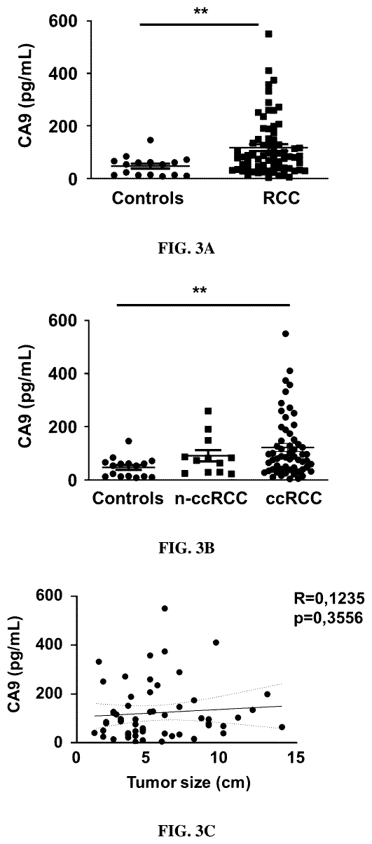

Levels of s-CA9 detected by ELISA were significantly higher in plasma from RCC patients (n=70) than in plasma from healthy controls (FIG. 3A). In particular, the plasma s-CA9 levels in ccRCC patients were significantly higher than in healthy controls. The mean of plasma s-CA9 levels were 117.5 pg/mL (range 5-550.29) in ccRCC patients, 91.2 pg/mL in n-ccRCC patients, and 47.12 pg/mL in controls (FIG. 3B).

However, the plasma s-CA9 levels detected by ELISA did not correlate with the tumor size measured at pathologic examination in ccRCC patients (FIG. 3C), with the ISUP grade or the pathologic stage (Table 3). This contrasts with the data obtained in Example 1 where CA9⁺ MVs correlated with tumor size (FIG. 1E) and with ISUP grade I-II versus III-IV (Table 2).

TABLE 3
Relationship between s-CA9 levels measured by ELISA in plasma
and clinico-pathological variables.

| | ccRCC | ccRCC | | ccRCC | ccRCC | |
|---|---|---|---|---|---|---|
| | TNM | TNM | | ISUP | ISUP | |
| | stage | stage | p- | grade | grade | p- |
| | T1-T2 | T3-T4 | value | I-II | II-III | value |
| Case | 31 | 27 | | 32 | 25 | |
| number | | | | | | |
| Mean | 112 | 135.6 | 0.47 | 140.2 | 103.9 | 0.41 |
| (pg/mL) | | | | | | |
| Range | 3.82-357.5 | 16.18-550.3 | | 3.82-550.3 | 5-410.8 | |
| (pg/mL) | | | | | | |

ccRCC: clear cell renal cell carcinoma; TNM: (T) tumor, (N) node, (M) metastasis; ISUP: international society of urological pathology

### Example 3: Robustness of Circulating CA9⁺ MVs as a Diagnostic Tool in ccRCC

### Methods

### Receiver Operating Characteristic (ROC) Curves

To evaluate the diagnostic performance of CA9⁺ MVs between ccRCC and healthy controls, receiver operating characteristic (ROC) curves were plotted and other diagnostic characteristics such as sensitivity, specificity, positive predictive value (PPV), negative predictive value (NPV), Yule's Q coefficient, Youden's index and the Chi² test of significant variables were calculated.

### Results

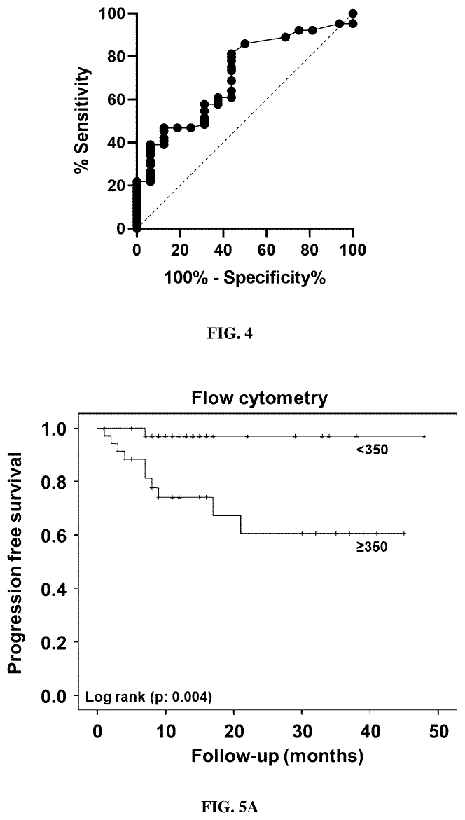

In order to test the robustness of flow cytometry as diagnostic tool for detecting CA9⁺ MVs in ccRCC, we generated a ROC curve (FIG. 4). Cut-off values for CA9⁺ MVs detected by flow cytometry to predict ccRCC were derived from ROC curves in 64 patients. CA9⁺ MVs>1.85% showed an area under the curve (AUC) of 0.70 (95% CI: 0.57-0.84) and a sensitivity of 68.8%, specificity of 60.9%.

Of the 64 ccRCC patients, 88.6% (PPV) of individuals who achieve a higher cut-off of 1.85% on the flow cytometry are accurately diagnosed with ccRCC. Conversely, 30.7% (NPV) of individuals who achieve a cut-off of 1.85% or lower are accurately diagnosed as healthy. Despite Youden's index was 0.3, Yule's Q coefficient was calculated as 0.55, indicating a strong association between CA9⁺ MVs detected by flow cytometry and ccRCC. In addition, the Chi² test result was ≤0.05 (Table 4), demonstrating that a threshold value of 1.85% could be considered predictive in these patients.

TABLE 4
Test characteristics of CA9⁺ MVs detected
by flow cytometry for prediction of ccRCC

| | CA9⁺ MVs |
|---|---|
| True positives | 39 |
| True negatives | 11 |
| False positives | 5 |
| False negatives | 25 |
| Sensitivity (95% CI) | 68.8 (41.3-88.9) |
| Specificity (95% CI) | 60.9 (47.9-72.9) |
| Positive predictive value (%) | 88.6 |
| Negative predictive value (%) | 30.7 |
| Yule's Q coefficient | 0.55 |
| Youden's index | 0.3 |
| Chi² test | ≤0.05 |
| Accuracy % | 62.5 |

### Example 4: Evaluation of Progression-Free Survival

### Methods

### Progression-Free Survival Prognosis

In addition to the percentage of CA9⁺ MVs, a numeration in absolute value per μL of plasma was also performed by flow cytometry on 76 patients using Flowcount beads. Association between clinical and pathological characteristics and the absolute number of CA9⁺ MVs detected by flow cytometry and s-CA9 concentration measured by ELISA is reported in Table 5. Here, the median values were used to divide the patients into high- and low-CA9 groups and to establish a threshold value. Progression-free survival (PFS) was estimated using the Kaplan-Meier method for patients without metastasis and comparison was performed by the log-rank test.

TABLE 5
RCC patient and tumor characteristics according to median levels of MVs carrying
CA9/μL plasma and s-CA9 measured by flow cytometry and ELISA, respectively.
The median was used to divide the patients into high- and low-CA9 groups.

| | n = 76 | | | n = 70 | | |
|---|---|---|---|---|---|---|
| | CA9-MVs | CA9-MVs | | S-CA9 | s-CA9 | |
| | <350 | ≥350 | P | <88 | ≥88 | P |
| Gender | | | 1 | | | 1 |
| Female | 13 | 11 | | 11 | 10 | |
| Male | 27 | 25 | | 26 | 23 | |
| Median age (Year, SD) | 62.8 (12) | 63.8 (11) | 0.717 | 62 (13) | 65 (11) | 0.275 |
| T Stage | | | 0.295 | | | 0.455 |
| 1 | 22 | 14 | | 19 | 16 | |
| 2 | 1 | 4 | | 4 | 1 | |
| 3 | 15 | 15 | | 11 | 15 | |
| 4 | 1 | 2 | | 1 | 1 | |
| Histological subtype | | | 1 | | | 0.23 |
| Clear renal cell | 33 | 30 | | 28 | 29 | |
| carcinoma | | | | | | |
| Others | 7 | 6 | | 37 | 33 | |
| ISUP grade | | | 0.834 | | | 0.681 |
| 1 | 3 | 2 | | 2 | 3 | |
| 2 | 19 | 14 | | 17 | 16 | |
| 3 | 10 | 11 | | 10 | 6 | |
| 4 | 6 | 7 | | 5 | 7 | |
| Metastasis | | | 0.117 | | | |
| 0 | 35 | 35 | | 33 | 32 | 1 |
| 1 | 4 | 0 | | 2 | 1 | |
| Median Tumor | 4.95 (2.5) | 6.26 (3.3) | 0.05 | 5.42 (2.9) | 5.42 (3) | 0.99 |
| size (cm, SD) | | | | | | |
| Recurrence | 1 | 11 | 0.006 | 3 | 7 | 0.139 |
| For patient without | | | | | | |
| metastasis at | | | | | | |
| diagnosis | | | | | | |

SD: Standard deviation

### Results

The median of the numbers of CA9⁺ MVs determined by flow cytometry was 350 MVs/μL of plasma (33-47328), whereas the median value of s-CA9 concentration quantified by ELISA was 88 pg/mL (4-550) (n=70). Tumor size (p=0.05) and tumor recurrence (p=0.006) were correlated with high values of CA9⁺ MVs/μL of plasma.

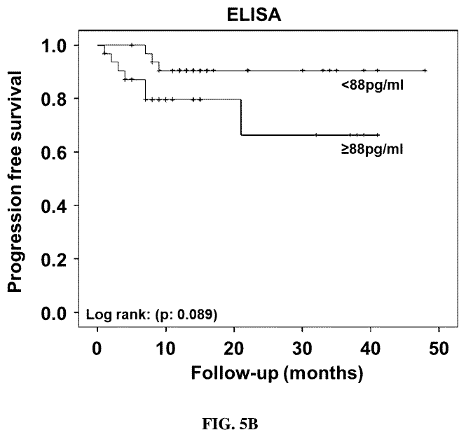

Patients with high number of CA9⁺ MVs (>350 CA9⁺ MVs/μL of plasma) had a lower progression-free survival (p=0.01) compared to patients with low CA9⁺ MV number (<350 CA9-MVs/μL of plasma) (FIG. 5A), whereas high concentrations of s-CA9 (>88 pg/mL) measured by ELISA did not significantly correlate with low progression-free survival (p=0.089) (FIG. 5B).

In conclusion, determining CA9⁺ MVs levels offers a robust prognostic of RCC recurrence, unlike s-CA9 concentration.

1-14. (canceled)

15. A method of predicting the risk of recurrence in a subject undergoing treatment for, or having undergone treatment for, clear cell renal cell carcinoma (ccRCC), comprising:
a) measuring the level of extracellular vesicles expressing carbonic anhydrase 9 (CA9⁺ EVs) in a sample previously obtained from the subject,
b) comparing the level of CA9⁺ EVs with a reference level,
c) assigning the subject to a high-risk group of ccRCC recurrence if the level of CA9⁺ EVs is substantially higher than the reference level, or assigning the subject to a low-risk group of ccRCC recurrence if the level of CA9⁺ EVs is substantially similar or lower than the reference level.

16. The method according to claim 15, wherein CA9⁺ EVs are microvesicles expressing carbonic anhydrase 9 (CA9⁺ MVs).

17. The method according to claim 15, wherein the level of CA9⁺ EVs is expressed as an absolute number of CA9⁺ EVs in a given volume of sample.

18. The method according to claim 17, wherein the absolute number of CA9⁺ EVs in a given volume of sample is determined by a method consisting of:
a) centrifuging the sample previously obtained from the subject at about 260 g for about 15 minutes,
b) centrifuging the supernatant retrieved after step a) at about 1500 g for about 20 minutes, and
c) measuring the absolute number of CA9⁺ EVs in a given volume of the supernatant retrieved after step b).

19. The method according to claim 15, wherein the reference level is derived from the measurement of CA9⁺ EVs in a sample from a reference subject or in samples from a population of reference subjects, said reference subject(s) being known to have low risks of ccRCC recurrence.

20. The method according to claim 16, wherein the reference level is about 350 CA9⁺ MVs/μL of sample.

21. A method of diagnosing clear cell renal cell carcinoma (ccRCC) in a subject or of identifying a subject as being at risk of developing ccRCC, comprising:
a) measuring the level of extracellular vesicles expressing carbonic anhydrase 9 (CA9⁺ EVs) in a sample previously obtained from the subject,
b) comparing the level of CA9⁺ EVs with a reference level,
c) concluding that the subject is affected with, or is at risk of developing, ccRCC if the level of CA9⁺ EVs is substantially higher than the reference level.

22. The method according to claim 21, wherein CA9⁺ EVs are microvesicles expressing carbonic anhydrase 9 (CA9⁺ MVs).

23. The method according to claim 21, wherein the level of CA9⁺ EVs is expressed as a percentage of CA9⁺ EVs out of the total extracellular vesicles in the sample.

24. The method according to claim 21, wherein the reference level is derived from the measurement of CA9⁺ EVs in a reference subject or in a population of reference subjects not suffering from and/or not diagnosed with ccRCC.

25. The method according to claim 22, wherein the reference level is 1.85% of CA9⁺ MVs out of the total microvesicles in the sample.

26. The method according to claim 21, wherein diagnosing ccRCC consists of determining the tumor size and/or grading of the ccRCC.

27. The method according to claim 15, wherein the sample is a blood sample.

28. The method according to claim 15, wherein measuring the level of CA9⁺ EVs is carried out by flow cytometry.

29. The method according to claim 21, wherein the sample is a blood sample.

30. The method according to claim 21, wherein measuring the level of CA9⁺ EVs is carried out by flow cytometry.

31. A method for treating clear cell renal cell carcinoma (ccRCC) in a subject in need thereof, said method comprising:
a) diagnosing clear cell renal cell carcinoma (ccRCC) in said subject or identifying said subject as being at risk of developing ccRCC, by
(1) measuring the level of extracellular vesicles expressing carbonic anhydrase 9 (CA9⁺ EVs) in a sample previously obtained from the subject,
(2) comparing the level of CA9⁺ EVs with a reference level,
(3) concluding that the subject is affected with, or is at risk of developing, ccRCC if the level of CA9⁺ EVs is substantially higher than the reference level; and
b) treating said subject affected with ccRCC, or identified as being at risk of developing ccRCC, for ccRCC.

32. The method according to claim 31, wherein CA9+ EVs are microvesicles expressing carbonic anhydrase 9 (CA9+ MVs).

33. The method according to claim 31, wherein the level of CA9⁺ EVs is expressed as a percentage of CA9⁺ EVs out of the total extracellular vesicles in the sample.

34. The method according to claim 31, wherein the sample is a blood sample.


SEQUENCE LISTING (1 sequence)

SEQ ID NO: 1 - protein, 459 aa, Homo sapiens
  Carbonic anhydrase 9 (CA9)
MAPLCPSPWLPLLIPAPAPGLTVQLLLSLLLLVPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA